(19) United States
(12) Patent Application Publication
(10) Pub. No.: US 2022/0241049 A1
(43) Pub. Date: Aug. 4, 2022

(54) TOOTH EXTRACTION GLOVE AND METHOD OF EXTRACTING TEETH

(71) Applicant: Jim Meletlidis, Dracut, MA (US)

(72) Inventor: Jim Meletlidis, Dracut, MA (US)

(21) Appl. No.: 17/248,593

(22) Filed: Jan. 29, 2021

(51) Int. Cl. A61C 3/14; A41D 19/015; A41D 19/00; A61B 42/20; A61B 42/10

(52) U.S. Cl. CPC A61C 3/14; A41D 19/01564; A41D 19/0051; A61C 2203/00; A61B 42/10; A41D 2400/80; A61B 42/20

(57) ABSTRACT

A tooth extraction glove is provided. The tooth extraction glove has finger coverings for only the index finger and thumb. Each finger covering has an aperture adjacent a distal end. The present invention also embodies separate mounting tips that cover the user's index finger and thumb tips, respectively. Each mounting tip provides one of a plurality of exchangeable prongs extending beyond the distal end of the mounting tip, wherein the prong slides through the above-mentioned aperture for gripping and extracting a tooth. The finger coverings provide decorative indicia to effectuate a non-threatening feel and appearance while the prongs extract the tooth.

### BACKGROUND OF THE INVENTION

The present invention relates to tools of the dental arts and, more particularly, a tooth extraction glove and method for parents and dentists to wiggle out baby teeth in a non-threating, efficient way.

As a general rule, children hate going to the dentist. The anxiety in anticipation of the expected pain, makes it difficult for the dentist to apply their trade. For similar reasons it is difficult for a dentist and/or parent to get a child to sit still when attempting to extract a loose tooth. Unfortunately, there are no non-scary looking tools for extracting children's teeth to facilitate such a benign process, especially tools that alleviate the anxiety of the child.

For dentists, scary-looking anxiety-heightening forceps are the only option. For parents, no real dependable device is available for home use.

As can be seen, there is a need for a tooth extraction glove and method for parents and dentists to wiggle out baby teeth in a non-threating, efficient way. The present invention embodies a wearable tool with a non-threating appearance and reusable, interchangeable parts that can save a parent time and money by not having to take their child to the dentist. Or, if the parent does take the child to the dentist, it can help a dentist because the present invention is embodied in a playful- looking article which can calm children prior to and during the extraction of the tooth.

### SUMMARY OF THE INVENTION

In one aspect of the present invention, a tooth extraction tool including the following: a partial hand covering adapted to fit over the hand of a user, a thumb finger covering extending from said partial hand covering adapted for covering a thumb of the hand of the user; an index finger covering extending from said partial hand covering adapted for covering an index finger of the hand of the user; and an aperture along a distal end of each finger covering, wherein a prong extends out of each aperture.

In another aspect of the present invention, the tooth extraction tool includes a thumb mounting tip adapted to fit over the tip of said thumb, wherein the prong removably attaches thereto; an index finger mounting tip adapted to fit over the tip of said index finger, wherein the prong removably attaches thereto; a sleeve provided by the thumb mounting tip, wherein the sleeve removably receives said prong of a plurality of interchangeable prongs of different lengths; a sleeve provided by the index finger mounting tip, wherein the sleeve removably receives said prong of a plurality of interchangeable prongs of different lengths; a textured surface along a portion of said prong, wherein the partial hand covering has a palm side and a back side, said partial hand covering having an opening so that skin of the middle finger, the ring finger and the little finger is exposed; and one or more decorative indicia along the palm side and the back side.

These and other features, aspects and advantages of the present invention will become better understood with reference to the following drawings, description and claims

### BRIEF DESCRIPTION OF THE DRAWINGS

FIG. 1 is a side perspective view of an exemplary embodiment of the present invention, shown in use;

FIG. 2 is an exploded side elevation view of an exemplary embodiment of the present invention;

FIG. 3 is a detail front view of an exemplary embodiment of a tip component of the present invention, as indicated by line 3-3 in FIG. 2;

FIG. 4 is a top plan view of an exemplary embodiment of the present invention on a right-hand of a user;

FIG. 5 is a top plan view of an exemplary embodiment of the present invention on a left-hand of a user; and

FIG. 6 is a side perspective view of an exemplary embodiment of the present invention, shown in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a tooth extraction glove having finger coverings for only the index finger and thumb. Each finger covering has an aperture adjacent a distal end. The present invention also embodies separate mounting tips that cover the user's index finger and thumb tips, respectively. Each mounting tip provides one of a plurality of exchangeable prongs extending beyond the distal end of the mounting tip, wherein the prong slides through the above-mentioned aperture for gripping and extracting a tooth. The finger coverings provide decorative indicia to effectuate a non-threatening feel and appearance while the prongs extract the tooth.

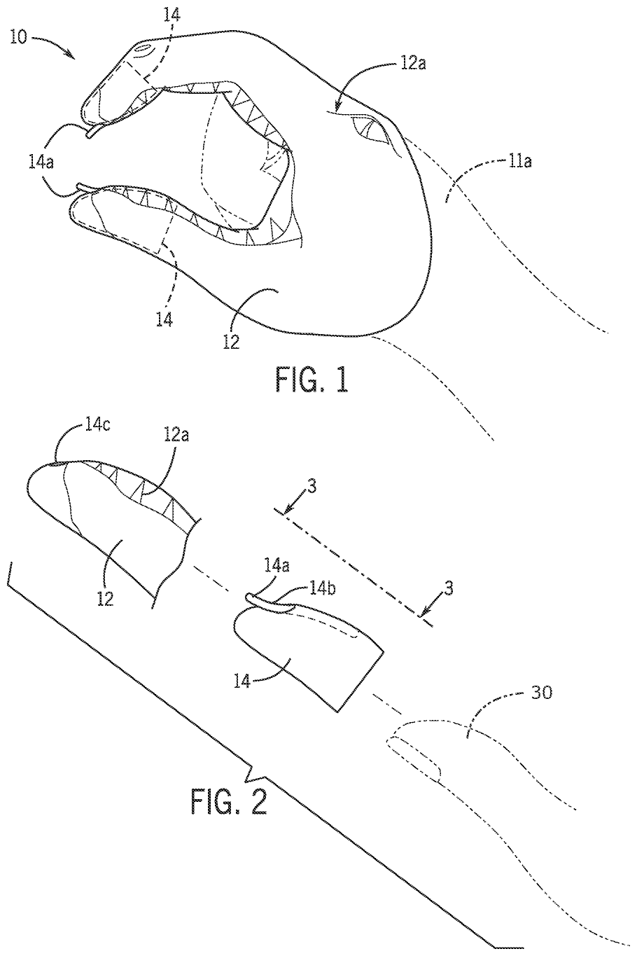

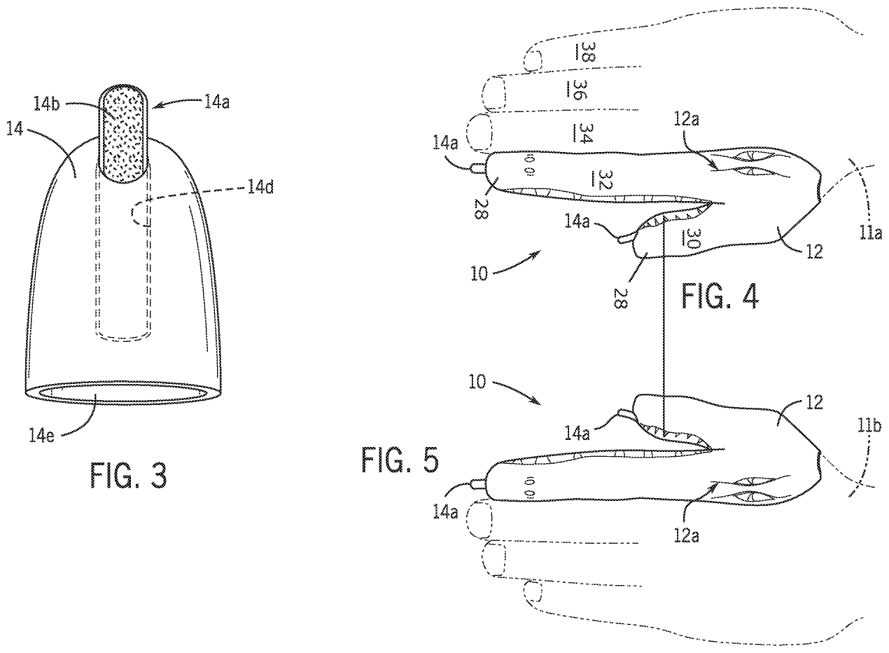

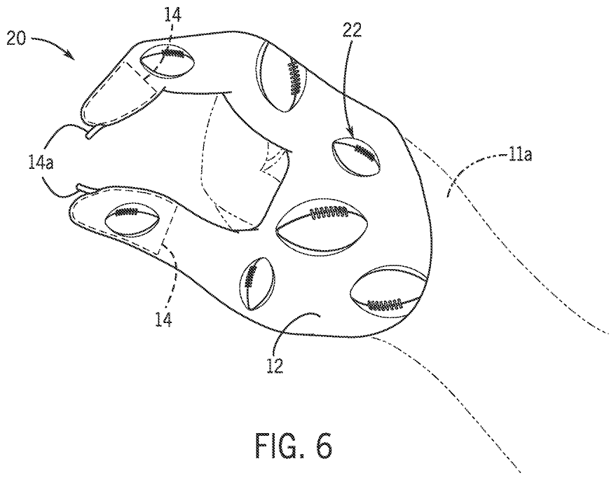

Referring now to FIGS. 1 through 6, the present invention may include a tooth extraction glove 10 or 20 having a partial hand covering 12 with finger coverings 28 for the thumb 30 and the index finger 32 and without finger coverings for the middle finger 34, the ring finger 36, and the little finger 38. The finger coverings 28 have an aperture 14c at or adjacent to their respective distal end. The partial hand covering 12 may be dimensioned and adapted to be worn by a human user on either their right hand 11a or their left hand 11b.

The tooth extraction glove 10 or 20 may also include mounting tips 14 dimensioned and adapted to slide on to the tip of a human thumb 30 or index finger 32 by way of a fingertip opening 14e.

Each mounting tip 14 has a prong 14a that protrudes beyond a distal end thereof. The prong 14a is dimensioned and adapted to slide through the aperture 14c of the finger coverings 28 when both the mounting tip 14a and the main covering 12 are simultaneously worn. Each prong 14a may be removably secured in a sleeve 14d of the mounting tip 14, thereby different prongs 14a of different lengths and/or widths can be utilized in the same mounting tip 14.

The mounting tips 14 may be made from any durable material, including but not limited to silicone, other plasticized materials, or synthetic materials. Each prong 14a may be made from stainless-steel or durable plasticized materials, or metallic materials. Each prong 14 may have serrated edges, gritted/patterned belly and/or a textured surface 14b for added grip. The textured surface 14b may extend along a distal portion of the prong 14a. The prong 14a may have a curvature to facilitate entering a cavity between a crown of a tooth and its associated gums. The textured surface 14b may be along this curvature.

The partial hand covering 12 may be nylon, non-latex, or the like and provide decorative indicia 12a and 22, especially along opposing surfaces of the thumb 30 and the index finger 32 to effectuate an appearance or look of a mouth of animal or other creature to evoke a playfulness in a child. The decorative indicia 12a and 22 may also be adapted to effectuate a non-threatening, anxiety-lessening appearance and overall look and feel, especially to smaller children.

A method of using the present invention may include the following. The tooth extraction glove 10 or 20 disclosed above may be provided. Dependent on the position of the tooth one wishes to extract (front vs back), the user inserts the appropriate-sized prongs 14a into the mounting tips 14, dons said mounting tips on the hand of their choice, and then the user puts one the partial hand covering 12 so that said prongs 14a slide through the apertures 14c. Carefully, the user can insert the two opposable prongs 14a of the thumb 30 and the index finger 32 to engage and wiggle, pincer-like, the loose tooth until extracted. Place gauze when finished. Rinse the tooth and prepare it for the tooth fairy!

It should be understood, of course, that the foregoing relates to exemplary embodiments of the invention and that modifications may be made without departing from the spirit and scope of the invention as set forth in the following claims.

What is claimed is:

1. A tooth extraction tool, comprising:
a partial hand covering adapted to fit over the hand of a user, a thumb finger covering extending from said partial hand covering adapted for covering a thumb of the hand of the user;
an index finger covering extending from said partial hand covering adapted for covering an index finger of the hand of the user; and
an aperture along a distal end of each finger covering, wherein a prong extends out of each aperture.

2. The tooth extraction tool of claim 1, further comprising:
a thumb mounting tip adapted to fit over the tip of said thumb, wherein the prong removably attaches thereto.

3. The tooth extraction tool of claim 1, further comprising:
an index finger mounting tip adapted to fit over the tip of said index finger, wherein the prong removably attaches thereto.

4. The tooth extraction tool of claim 2, further comprising:
a sleeve provided by the thumb mounting tip, wherein the sleeve removably receives said prong of a plurality of interchangeable prongs of different lengths and/or widths.

5. The tooth extraction tool of claim 3, further comprising:
a sleeve provided by the index finger mounting tip, wherein the sleeve removably receives said prong of a plurality of interchangeable prongs of different lengths and/or widths.

6. The tooth extraction tool of claim 4, further comprising a textured surface along a portion of said prong.

7. The tooth extraction tool of claim 5, further comprising a textured surface along a portion of said prong.

8. The tooth extraction tool of claim 1, wherein the partial hand covering has a palm side and a back side, said partial hand covering having an opening so that skin of the middle finger, the ring finger and the little finger is exposed.

9. The tooth extraction tool of claim 8, further comprising one or more decorative indicia along the palm side and the back side.
